(19) United States
(12) Patent Application Publication
(10) Pub. No.: US 2025/0268943 A1
(43) Pub. Date: Aug. 28, 2025

(54) STEM CELL-DERIVED EXTRACELLULAR VESICLES AND METHODS OF USE THEREOF

(71) Applicant: Washington University, St. Louis, MO (US)

(72) Inventor: Hua Shen, St. Louis, MO (US)

(21) Appl. No.: 19/096,315

(22) Filed: Mar. 31, 2025

Related U.S. Application Data
(62) Division of application No. 17/294,006, filed on May 14, 2021.
(60) Provisional application No. 62/767,866, filed on Nov. 15, 2018.

(51) Int. Cl. A61K 35/28; A61L 27/38; B82Y 5/00; C12N 5/0775; C12N 5/0786

(52) U.S. Cl. CPC A61K 35/28; A61L 27/3834; C12N 5/0645; C12N 5/0667; B82Y 5/00

(57) ABSTRACT

Disclosed herein are bionanoparticles of adipose-derived stem cell extracellular vesicles, a tissue repair matrix comprising the bionanoparticles, and methods of use thereof for enhanced tendon healing.

### CROSS REFERENCE TO RELATED APPLICATIONS

This application is a divisional application based on U.S. application Ser. No. 17/294,006, filed May 14, 2021 which claims the benefit of International Patent Application No. PCT/US2019/061746, filed Nov. 15, 2019, which claims priority to U.S. Provisional Application No. 62/767,866, filed Nov. 15, 2018, the contents of which are entirely incorporated by reference herein.

### GOVERNMENT INTEREST STATEMENT

This invention was made with government support under TR002345, AR062947 and AR075274 awarded by the National Institutes of Health. The government has certain rights in the invention.

### FIELD

The present invention relates stem cell-derived extracellular vesicles and methods of use thereof.

### BACKGROUND

Although the immediate causes and affected tendons of various tendon injuries are different, they all mostly affect relative young and otherwise healthy persons and yet with today's advanced surgical techniques and rehabilitation approaches, a delay or failure in return to pre-injury activity remains the major challenge for orthopedic surgeons. The unsatisfactory functional outcomes mainly result from (1) excessive inflammation that causes cell death and matrix degradation and (2) incompetent tendon matrix regeneration, thus impeding tendon structure and strength recovery.

Accordingly, there is a need for biologically-based therapies to fully restore tendon structure and function.

### BRIEF SUMMARY

The disclosure provides for a bionanoparticle for enhanced musculoskeletal healing in a patient that includes an isolated extracellular vesicle (EV) from adipose-derived stem cells (ASCs). In an aspect, the EVs are iEVs from interderon gamma (IFNγ)-primed ASCs. In an aspect, the ASCs are isolated from the patient or may be isolated from another individual. The patient may be a human or another animal such as a cat, dog, horse, or any other mammal. In another aspect, the bionanoparticle further includes small RNAs, such as microRNAs and messenger RNAs (mRNAs) within the EV or iEV.

Further provided herein is a tissue repair matrix including a collagen sheet and a plurality of bionanoparticles of ASC EVs or iEVs loaded within the collagen sheet.

Also provided herein is a method of preparing a tissue repair matrix. The method may include harvesting a plurality of ASCs, isolating a plurality of EVs from the ASCs, and loading a collagen sheet with the plurality of EVs. The method may further include further comprising priming the ASCs with inflammatory cytokines, for example, IFNγ.

The disclosure further provides for a method for treating an injured tissue. The method may include comprising applying a plurality of bionanoparticles to the injured tissue. In an aspect, the bionanoparticles may be applied directly to the injured tissue. In another aspect, the bionanoparticles may be injected subcutaneously, peritendinously, or intraarticularly near the injured tissue. In another aspect, the method for treating an injured tissue may include applying a tissue repair matrix to the injured tissue. In an aspect, the injured tissue may be musculoskeletal tissue or soft tissue, for example a tendon. In an aspect, the tissue repair matrix may be applied during operative repairs. In another aspect, the tissue repair matrix is placed on top the injured tissue. In other aspects, the tissue repair matrix surrounds the injured tissue. In some aspects, the tissue repair matrix is attached to the injured tissue with or without suturing. In various aspects, the ASC EVs or iEVs attenuate inflammatory NFκB activity in the injured tissue. In another aspect, the ASC EVs or iEVs promote tendon matrix regeneration in the early phase of tendon healing.

Additional aspects and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

### DESCRIPTION OF THE DRAWINGS

The patent or application file contains at least one drawing executed in color. Copies of this patent or patent application publication with color drawing(s) will be provided by the Office upon request and payment of the necessary fee.

The description will be more fully understood with reference to the following figures, which are presented as variations of the disclosure and should not be construed as a complete recitation of the scope of the disclosure, wherein:

FIG. 1 illustrates the regulatory function of ASCs.

FIG. 2A shows representative immunofluorescence images of mouse ASCs stained with antibodies specific for the mesenchymal stem cell markers CD29, CD44, and CD90, respectively. The stem cell markers are stained in green and the nuclei of ASCs are stained in blue.

FIG. 2B shows representative transmission electron microscopy images of mouse ASC EVs released by naïve (EV) and IFNγ-primed ASCs (iEV) along with the EV-free EV collection medium.

FIG. 2C shows western blots that detect exosome markers CD9 and CD63 in isolated ASC EVs. STD, size standard. MW, molecular weight.

FIG. 3 shows the induction of macrophage polarization.

FIG. 4 shows ASCs promote macrophage M2 polarization.

FIG. 5A, FIG. 5B, and FIG. 5C show characterization of mouse ASC EVs.

FIG. 6A and FIG. 6B show the effect of ASC EVs on macrophages.

FIG. 7 shows the application of ASC EVs in tendon repair.

FIG. 8A shows the application and biodistribution of ASC EVs in mice Achilles tendon after injury.

FIG. 8B and FIG. 8C are representative whole-mount fluorescence images showing the injury site of a mouse Achilles tendon 7 days after partial transection and application of ASC EVs. Scale bar=100 μm.

FIG. 8D, FIG. 8E, and FIG. 8F are fluorescence images showing the sagittal section of the tendon shown in FIG. 8B and FIG. 8C at the boxed region. The residing tenocytes expressed ScxGFP (in green). All cells were counterstained with 4′,6-diamidino-2-phenylindole (DAPI, in blue). The arrow heads point to the EV positive signals at the DAPI positive and ScxGFP negative cells. The arrows point to the EV positive signals at the DAPI and ScxGFP double positive tenocytes. Scale bar=50 μm.

FIG. 9A shows representative bioluminescence images of the changes in nuclear factor-κB (NF-κB) activity at the repair site of NF-κB-GFP-luciferase (NGL) NF-κB reporter mice prior to (Pre) and at the indicated time points after right Achilles tendon repair and indicated treatments.

FIG. 9B is a graph quantifying the changes in nuclear factor-κB (NF-κB) activity at the repair site of NF-κB-GFP-luciferase (NGL) NF-κB reporter mice prior to (Pre) and at the indicated time points after right Achilles tendon repair and indicated treatments. * and-p<0.05 by Student-Newman-Keuls (SNK) multiple comparisons, compared with the pre-injury level of respective group and between indicated groups, respectively.

FIG. 10 shows matrix remodeling in pentachrome-stained Achilles tendon sections 14 days after tenotomy and indicated treatments. All mice were subjected to right Achilles tenotomy and left Achilles sham surgery.

FIG. 11A shows fold changes in the messenger RNA (mRNA) expression levels of Ifng in mouse Achilles tendons 7 days after tendon repair and indicated treatments. *p<0.05 compared with contralateral intact tendons by paired t test; \hat{ }p<0.05 by one-way analysis of variance (ANOVA).

FIG. 11B shows fold changes in the messenger RNA (mRNA) expression levels of Nos2 in mouse Achilles tendons 7 days after tendon repair and indicated treatments. *p<0.05 compared with contralateral intact tendons by paired t test.

FIG. 11C shows fold changes in the messenger RNA (mRNA) expression levels of Tnf in mouse Achilles tendons 7 days after tendon repair and indicated treatments. *p<0.05 compared with contralateral intact tendons by paired t test.

FIG. 11D shows fold changes in the messenger RNA (mRNA) expression levels of II6 in mouse Achilles tendons 7 days after tendon repair and indicated treatments. *p<0.05 compared with contralateral intact tendons by paired t test.

FIG. 11E shows fold changes in the messenger RNA (mRNA) expression levels of II1b in mouse Achilles tendons 7 days after tendon repair and indicated treatments. \hat{ }p<0.05 by one-way analysis of variance (ANOVA). -p<0.05 between the indicated groups by Dunn's test.

FIG. 12A shows fold changes in the messenger RNA (mRNA) expression levels of Col1a1 and Col3a1 in mouse Achilles tendons 7 days after tendon repair and indicated treatments. *p<0.05 compared with paired uninjured tendons by paired t test.

FIG. 12B shows the relative messenger RNA (mRNA) abundance of Mmp1 in mouse Achilles tendons 7 days after tendon repair and indicated treatments. \hat{ }p<0.05 by one-way analysis of variance (ANOVA). -p<0.05 between the indicated groups by Tukey's test.

FIG. 12C shows fold changes in the messenger RNA (mRNA) expression levels of Scx and Tnmd in mouse Achilles tendons 7 days after tendon repair and indicated treatments. *p<0.05 compared with paired uninjured tendons by paired t test.

FIG. 12D shows changes in the relative messenger RNA (mRNA) abundance of Col2a1 and Sox9 in mouse Achilles tendons 7 days after tendon repair and indicated treatments. *p<0.05 compared with paired uninjured tendons by paired t test; \hat{ }p<0.05 by one-way analysis of variance (ANOVA). -p<0.05 between the indicated groups by Tukey's or Dunn's test (when appropriate).

FIG. 12E shows fold changes in the messenger RNA (mRNA) expression levels of Mmp13 and Mmp3 in mouse Achilles tendons 7 days after tendon repair and indicated treatments. *p<0.05 compared with paired uninjured tendons by paired t test.

FIG. 13A shows ASC EVs reduce macrophage NFκB activity induced by IL-1β.

FIG. 13B shows ASC EVs increase tenocyte proliferation. -p<0.05 between the indicated groups by t test.

FIG. 14A shows a comparison of gap and rupture rates of injured Achilles tendons from Repair, +EV, and +iEV groups. -p<0.05 between the indicated groups by an N−1 χ2 test.

FIG. 14B shows a comparison of the percentage of collagen-stained areas within the injured Achilles tendons from the indicated groups. \hat{ }p<0.05 by one-way analysis of variance (ANOVA). -p<0.05 between the indicated groups by Tukey's test.

FIG. 14C shows a representative image of pentachrome-stained coronal sections an intact Achilles tendon. The yellow dotted line delineates the boundary between Achilles tendon and the surrounding paratenon tissue. The black brace encloses the intact Achilles tendon, and the gray brace encloses the paratenon region of Achilles tendon.

FIG. 14D shows a representative image of pentachrome-stained coronal sections of a partially transected Achilles tendon treated with control repair only. The yellow dotted line delineates the boundary between the Achilles tendon and the surrounding paratenon tissue, and the white dotted line delineates the boundary between the intact and the transected portions of repaired Achilles tendon. The black brace encloses the intact portion of the Achilles tendon, the dotted black brace encloses the transected portion of repaired Achilles tendon, and the gray brace encloses the paratenon region of Achilles tendon.

FIG. 14E shows a representative image of pentachrome-stained coronal sections of a partially transected Achilles tendon treated with EVs from naïve ASCs. The yellow dotted line delineates the boundary between the Achilles tendon and the surrounding paratenon tissue, and the white dotted line delineates the boundary between the intact and the transected portions of repaired Achilles tendon. The black brace encloses the intact portion of the Achilles tendon, the dotted black brace encloses the transected portion of repaired Achilles tendon, and the gray brace encloses the paratenon region of Achilles tendon.

FIG. 14F shows a representative image of pentachrome-stained coronal sections of a partially transected Achilles tendon treated with iEVs. The yellow dotted line delineates the boundary between the Achilles tendon and the surrounding paratenon tissue, and the white dotted line delineates the boundary between the intact and the transected portions of repaired Achilles tendon. The black brace encloses the intact portion of the Achilles tendon, the dotted black brace encloses the transected portion of repaired Achilles tendon, and the gray brace encloses the paratenon region of Achilles tendon.

FIG. 15A is a representative superimposed fluorescence and bright field image of isolated NF-κB-GFP-luciferase (NGL) macrophages co-cultured with fluorescently labeled EVs (in white).

FIG. 15B is a representative superimposed fluorescence and bright field image of isolated NF-κB-GFP-luciferase (NGL) (A, B) macrophages co-cultured with fluorescently labeled iEVs (in white).

FIG. 15C is a representative superimposed fluorescence and bright field images of isolated FVB macrophages co-cultured with fluorescently labeled EVs (in white). Scale bar=50 μm applied to FIGS. 15A-15C.

FIG. 15D shows changes in nuclear factor-κB (NF-κB) activity in macrophages pre-treated with control medium (Medium), EV-free conditioned medium from naïve ASCs (+CM), EV-free conditioned medium from primed ASCs (+iCM), EVs from naïve ASCs in control medium (+EV), or EVs from primed ASCs in control medium (+iEVs) 6 h after interleukin-1β (IL-1β) treatment (5 ng/ml). \hat{ }p<0.05 by one-way analysis of variance (ANOVA). -p<0.05 between the indicated groups by Tukey's test.

FIG. 15E shows changes in NF-κB-responsive luciferase transgene (Nfkb-Luc) expression in isolated macrophages pre-treated with control medium (Medium), EVs (+EVs) or iEVs (+iEV) 24 h after IL-1β treatment (10 ng/ml). \hat{ }p<0.05 by one-way analysis of variance (ANOVA). -p<0.05 between the indicated groups by Tukey's test.

FIG. 16A shows EVs generated by ASCs that modify target cell (yellow circles) functions via intracellular delivery of regulatory molecular cargos.

FIG. 16B shows ASC EVs carry biologically enriched microRNA cargos that target macrophage inflammatory response.

FIG. 17 illustrates administration of ASC EVs through local injection or a collagen sheet wrapped around a repaired tendon during surgery.

FIG. 18 shows an EV loaded collagen sheet (exo-sheet) wrapped around a repaired tendon during surgery.

### DETAILED DESCRIPTION

The stem cell-derived extracellular vesicles and method of use will be understood, both as to its structure and operation, from the accompanying drawings, taken in conjunction with the accompanying description. It is noted that, for purposes of illustrative clarity, certain elements in various drawings may not be drawn to scale. Several variations of the device are presented herein. It should be understood that various components, parts, and features of the different variations may be combined together and/or interchanged with one another, all of which are within the scope of the present application, even though not all variations and particular variations are shown in the drawings. It should also be understood that the mixing and matching of features, elements, and/or functions between various variations is expressly contemplated herein so that one of ordinary skill in the art would appreciate from this disclosure that the features, elements, and/or functions of one variation may be incorporated into another variation as appropriate, unless described otherwise.

Adult adipose-derived stem cells (ASCs) may curb tendon inflammatory response and support tendon matrix regeneration. Compared to similar mesenchymal stem cells (MSCs) from other tissues (e.g. bone marrow, umbilical cord, etc.), ASCs are abundant in adults and may be obtained from liposuction waste and expanded quickly in culture. The application of ASCs in tendon repair, however, has been hindered by quite a few barriers. The dense extracellular matrix, small cross-sectional area, and restricted peripheral space of tendon tissue all limit sufficient cells to be delivered to the repair site. A synthetic scaffold system was generated to introduce ASCs into tendon stumps; however, the scaffold itself was found to pose additional structural and biological stress to injured tendons and therefore negated the therapeutic effects of ASCs. A biocompatible cell sheet was subsequently created to apply ASCs to the repair surface. The approach successfully restrained tendon inflammatory response and yet there was a lack of ASCs at the repair center to adequately improve tendon matrix regeneration. Additional issues, including the long-term bio-safety of stem cells (e.g. tumorigenicity, undesired spontaneous differentiation), further negatively impact the clinical translation of ASCs in tendon repair. Therefore, it is critical to develop a novel ASC-based therapeutic approach, which is capable of overcoming these translational issues and yet retaining all therapeutic benefits provided by ASCs, to effectively enhance tendon healing.

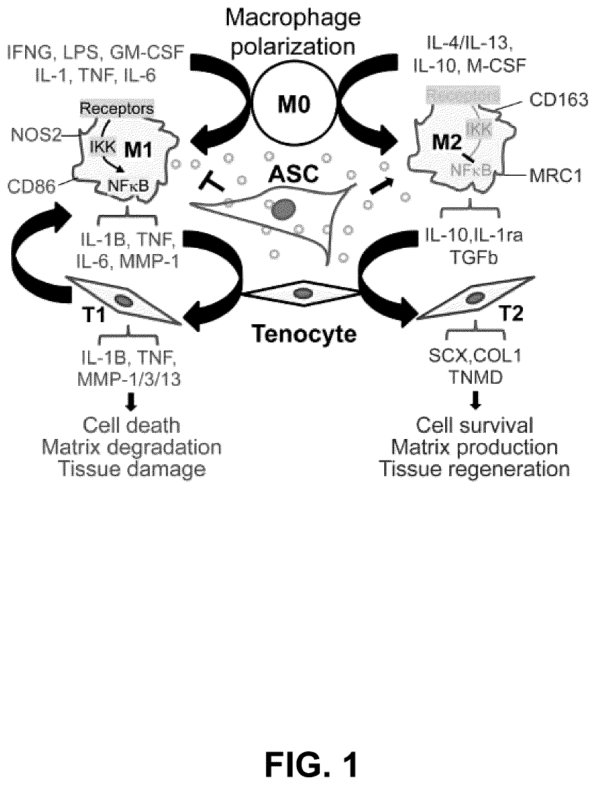

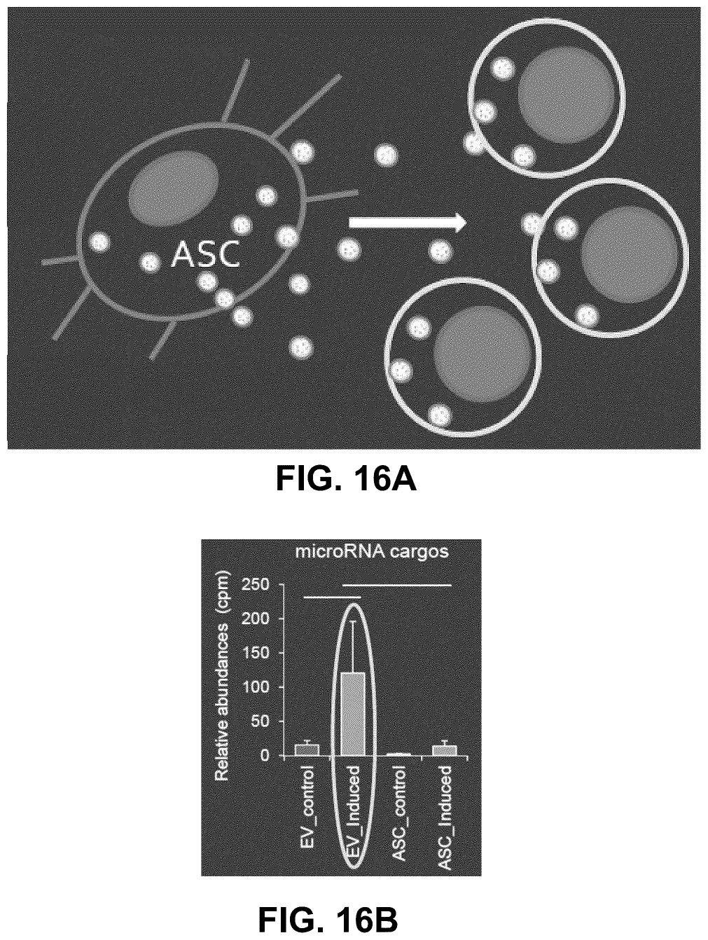

ASCs regulate tendon inflammatory response by promoting an anti-inflammatory and pro-regenerative M2 macrophage phenotype, which in turn facilitates regenerative healing (FIG. 1). The effect of ASCs relies on the secretory factors they release. Besides soluble molecules, ASCs release a large amount of extracellular vesicles (EVs). EVs are membrane surrounded structures that mediate cell-cell interaction via transferring functional molecules (e.g. mRNAs and microRNAs) to designated recipient cells (FIG. 16A). Although nearly all cells can produce EVs, the composition and function of EVs are cell-type specific. ASC EVs have been found to be taken up by macrophages and inhibit NFκB activities in activated macrophages in vitro and in repaired tendons in vivo. ASC EVs have also been found to be taken up by tenocytes and promote tenocyte proliferation in vitro and facilitate collagen regeneration in repaired tendons in vivo. Therefore, without being limited to a particular theory, ASCs may mainly function through Evs and Evs produced by ASCs may substitute ASCs as a novel therapeutic agent for tendon injuries. As EVs are nanosized and cell-free, they are more readily translatable and likely provide better therapeutic benefit.

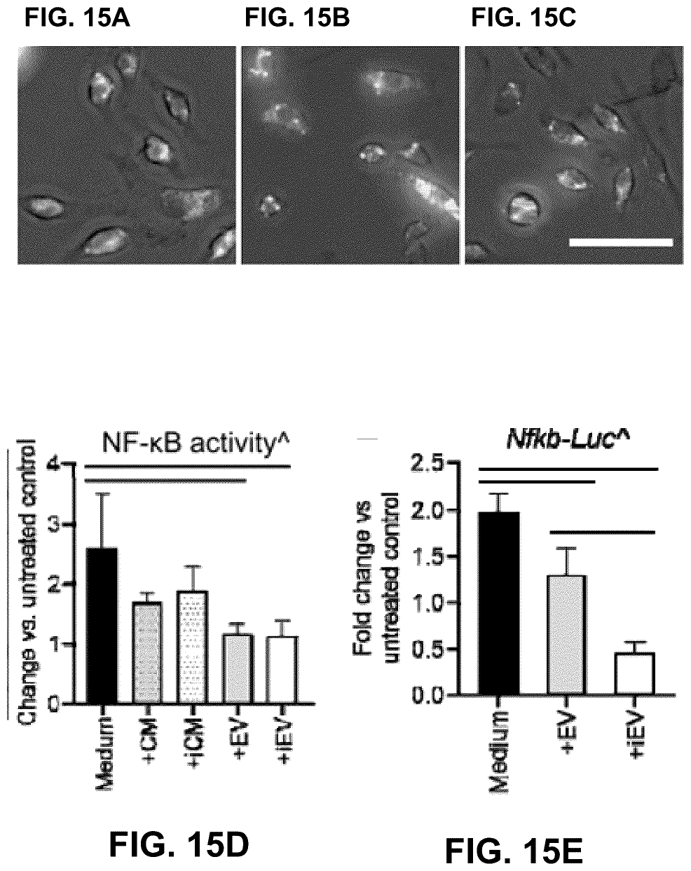

Like ASCs, ASC EVs may attenuate injury-induced tendon inflammatory response, including activation of NF-κB and induction of pro-inflammatory cytokine II1b and the major collagenase Mmp1 expression at an injury site. In addition to curbing inflammation, ASCs have also been found to facilitate regenerative healing by promoting collagen synthesis within the repair site. Likewise, ASC EVs may facilitate anabolic tissue response after injury, leading to increased collagen deposition at the center of tendon repair and reduced post-operative rupture/gap formation. Moreover, a side-by-side comparison between ASC-produced EVs and EV-free soluble factors demonstrates that the anti-inflammatory paracrine function of ASCs is primarily mediated by EVs (FIG. 15E).

Without being limited to any one theory, ASC EVs may facilitate early tendon healing due to the ability of ASC EVs to attenuate the macrophage inflammatory response. Specifically, ASC EVs primarily target infiltrating inflammatory cells at the site of tendon injury and subsequently reduce NF-κB activity and downstream II1b expression in injured tendons. In some examples, EVs can directly target macrophages and can block the inflammatory NF-κB signaling in these cells. In addition to macro-phages, some ASC EVs may be co-localized with tenocytes near the injury center and be taken up by tenocytes and promote tenocyte proliferation in culture. Therefore, ASC EVs may also directly facilitate residing tendon cell activity and function during tendon healing.

In some embodiments, EVs produced by inflammatory cytokine-primed ASCs (iEVs) may be more effective than EVs produced by naïve ASCs in curbing the inflammatory response in isolated macrophages and in repaired tendons. For example, EVs from IFNγ-primed ASCs have been found to be more potent in blocking macrophage inflammatory response in culture and in reducing inflammatory NF-κB activity and II1b gene expression in repaired tendons after acute Achilles tenotomy. The priming effect has been found to be associated with selective enrichment of certain regulatory miRNA cargos in primed EVs, such as miR-147, which is capable of inhibiting the macrophage inflammatory response. Additionally, priming may modify the cell and tissue selectivity and therefore the effects of EVs. The observed functional plasticity of ASCs and the potential dynamics of EV cargos introduce an opportunity to harness EV functions by controlling the biochemical environments of ASCs and, more directly, by controlling the active components of EV cargos.

The tendon is a fibrous tissue primarily made of collagen. At the gene expression level, iEV-treated tendons may express higher level of Col1a1, the primary tendon matrix gene; on the other hand, iEV-treated tendons may express lower level of Mmp1, which encode a protein that break down collagen. At the tissue level, iEV-treated tendons may produce a higher percentage of collagen at the midsubstance of injured tendon. iEV-treated tendons may show a substantially lower rupture/gap formation rate than did untreated tendons. Additionally, iEV-treated tendons may result in significant increases in the cartilage-related genes Col2a1 (16-fold) and Sox9 (four-fold) expression. While cartilage matrix formation is undesirable in normal tendon tissue, the increases after tendon injury and repair may be protective. Tendon injury may trigger substantial increases in Mmp13 (764-fold) and Mmp3 (37-fold) expression and both MMP3 and MMP13 preferentially degrade type II collagen and proteoglycan. Type II collagen and proteoglycan are constituents of tendon extracellular matrices. Therefore, the Sox9 and Col2a1 increases caused by iEV that counteract the Mmp13 and Mmp3 increases after tendon injury may be also beneficial for tendon healing.

While the regulatory function of stem cells offers great therapeutic opportunities in improving tendon and many other musculoskeletal conditions, their clinical application has been limited by many factors. Compared to current cell-and growth factor-based therapies, EVs have many advantages. First, EVs are highly translatable. The nanosized vesicles may be delivered in large quantity and can easily penetrate through biological barriers. Moreover, stem cell-EVs bind to collagen and therefore may be delivered locally via biocompatible collagen matrices. Importantly, the cell-free nature of EVs resolves the bio-safety concern of stem cells.

EVs enable a targeted and secured drug delivery. A cell-specific drug delivery may be desirable to maximize therapeutic efficacy and reduce side effects. EVs are natural nanocarriers, which are capable of cell-specific intracellular delivery via their surface markers. ASC EVs target both inflammatory macrophages and residing tenocytes and therefore are applicable to tendon injuries. Moreover, EVs, through the membrane structure, shield their cargos from various degrading enzymes, which are heavily present in injured tissues, and as a result, ensure the cargo integrity and bioactivity during delivery.

EVs are multifunctional. Unlike soluble factors that primarily act extracellularly, EVs can function both extracellularly through binding to extracellular matrices and surface receptors by membrane proteins and intracellularly through transferring cargo molecules across cell membrane. Not to mention, EVs carry a variety of molecular cargos with diverse biological functions. Besides the anti-inflammation function, ASC EVs have been found to facilitate tenocyte growth and proliferation and tendon matrix production as well. Therefore, EVs may be an effective and translatable stem cell-based therapy for tendon injuries and also provide a sophisticated solution for treating many other tendon and musculoskeletal disorders.

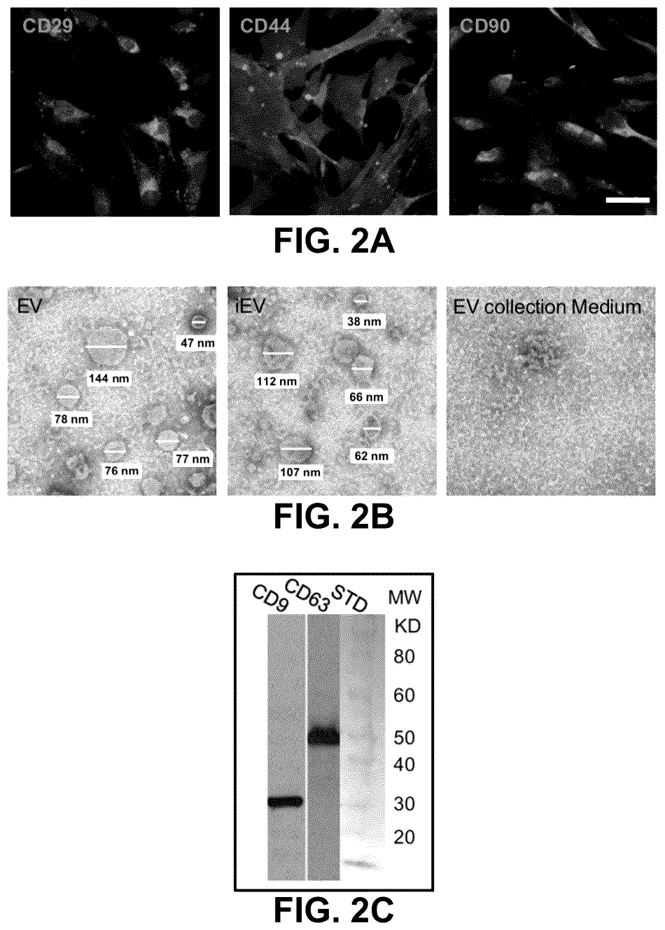

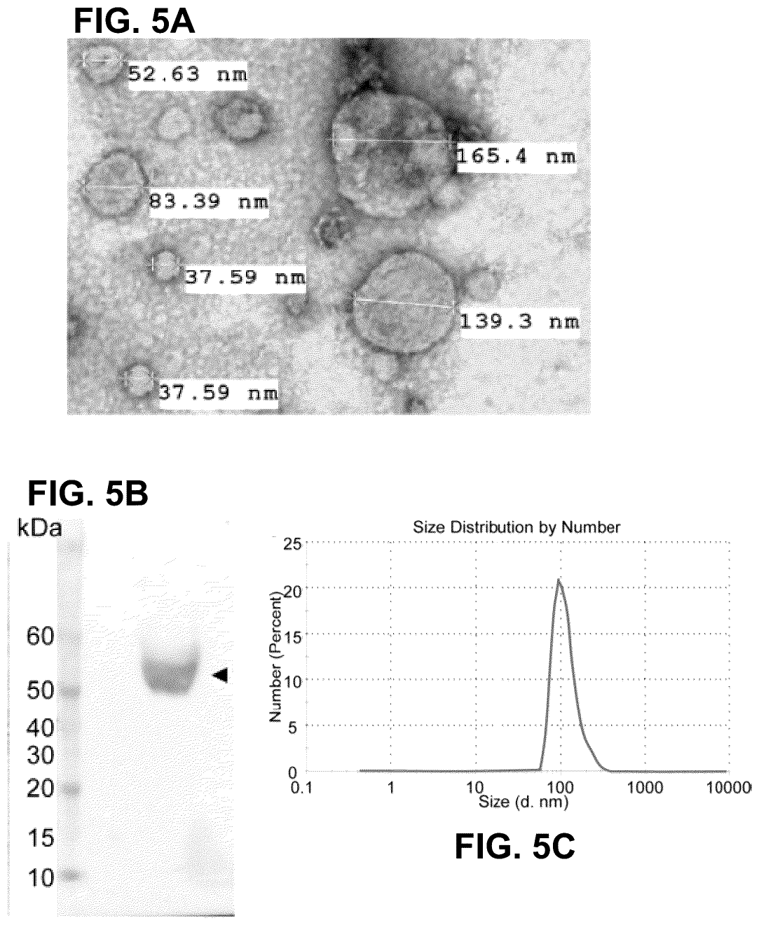

Provided herein are ASC EV-based therapeutic agents for enhanced musculoskeletal healing in a patient. In some embodiments, the EV's may be produced from IFNγ-primed ASCs (iEVs). In various embodiments, ASC EVs or iEVs may be used as stem cell-based therapeutic agents without cells. The EVs or iEVs may be isolated from ASCs and administered to a patient in need thereof as bionanoparticles. In some examples, the diameter of the EVs or iEVs may range from about 30 nm to about 200 nm (FIGS. 2B and 5A). In at least one example, the EVs or iEVs may have a peak diameter of about 116.7 nm (FIG. 5C). In other examples, the EVs or iEVs may express the exosome markers CD9 and CD63 (FIG. 2C). In various embodiments, the ASCs may be isolated from the patient or may be isolated from another individual. In various embodiments, the patient or other individual may be a human or another animal such as a dog, cat, horse, or any other mammal.

As seen in FIG. 16A, EVs generated by ASCs modify target cell (yellow circles) functions via intracellular delivery of regulatory molecular cargos. In some examples, the EVs or iEVs may have mRNA or microRNA cargos that target macrophage inflammatory response, as seen in FIG. 16B. In some embodiments, the bionanoparticles may further include mRNA or microRNA that modulate other cell activity and function including tenocyte proliferation and collagen production.

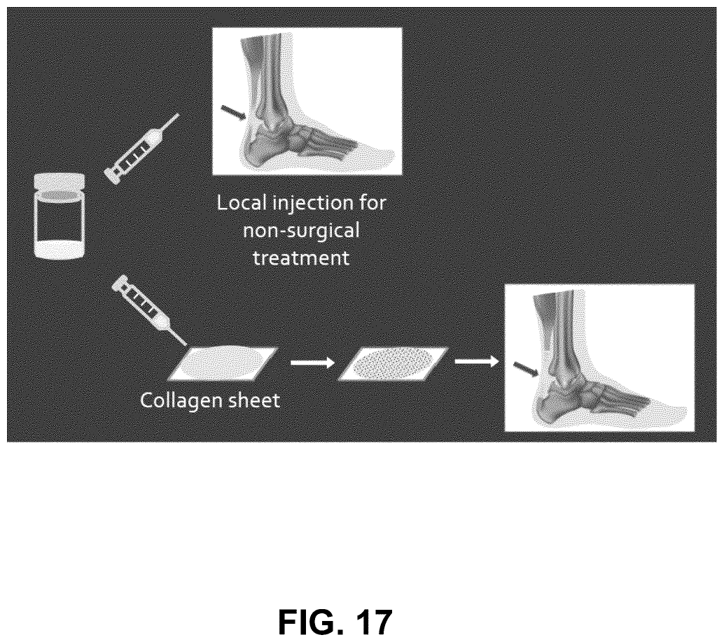

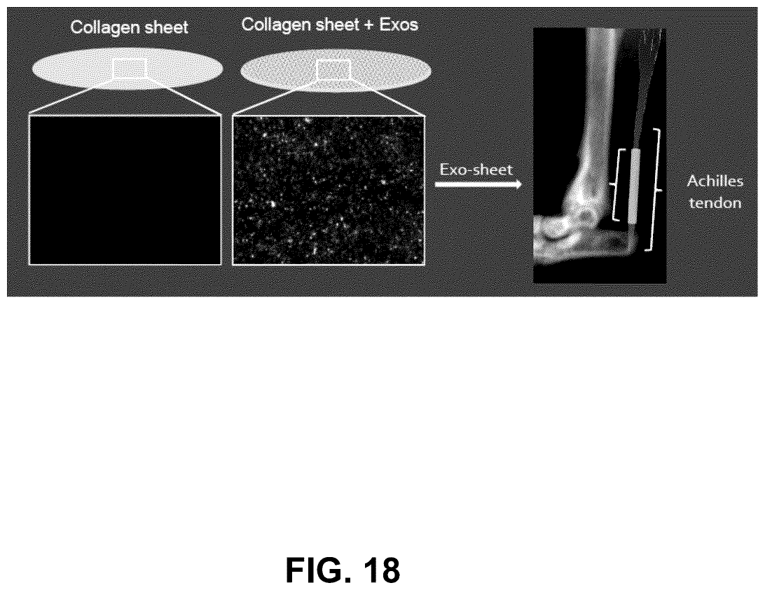

In some embodiments, bionanoparticles of ASC EVs or iEVs may be directly applied to a targeted tissue, injected (e.g. peritendinously, intraarticularly, or subcutaneously) near the targeted tissue, or be contained or loaded within a biocompatible matrix that is applied to the targeted tissue. In an example, the bionanoparticles may be applied as a local injection for non-surgical treatment, as seen in FIG. 17, or may be applied to a collagen sheet and wrapped around the tendon, as seen in FIGS. 17 and 18. In various embodiments, the ASC EVs or iEVs may be applied to a musculoskeletal or soft tissue injury. In an embodiment, the targeted tissue may be a tendon. For example, the tendon may be the Achilles tendon, patellar tendon, rotator cuff tendon, or flexor tendon.

In some embodiments, a biocompatible matrix may be loaded with the EVs or iEVs for application to a targeted tissue. The biocompatible matrix may include collagen. In one embodiment, the biocompatible matrix may be a collagen sheet. Without being limited to a particular theory, EVs or iEVs may bind to the collagen within the collagen sheet. In other embodiments, the biocompatible matrix may include other biocompatible polymers in addition to collagen, where the EVs or iEVs bind to the collagen within the matrix. A method of preparing a tissue repair matrix may include harvesting a plurality of ASCs, culturing and inducing the harvested ASCs, isolating a plurality of EVs from the ASCs, and loading a collagen sheet with the plurality of EVs. In some embodiments, the method may further include priming the ASCs with inflammatory cytokines, such as IFNγ or other stimuluses.

Provided herein are methods of using ASC EVs and iEVs to attenuate the repair site inflammatory response and facilitate tendon matrix regeneration in the earliest stage of tendon healing. Further provided herein are methods of retaining the exosomes in a biocompatible tissue repair matrix for targeted delivery of the exosomes for musculoskeletal tissue repair. In an example, the tissue repair matrix may be a collagen sheet (exo-sheet) that is loaded with a plurality of EVs. Retaining the EVs or iEVs locally via a biocompatible collagen sheet may improve exosome uptake by targeted tissues by allowing the EVs or iEVs to be retained locally near the targeted tissue. The resulting exo-sheet may be administrated during operative repairs and therefore expand the therapeutic applications of exosomes from non-operative to operative repairs. In some embodiments, the tissue repair matrix may be placed on top the injured tissue, surround the injured tissue, and/or be attached to the injured tissue with or without suturing.

Also provided herein is a method for treating an injured tissue by applying to the tissue a plurality of EV or iEV bionanoparticles or a tissue repair matrix with the EV or iEV bionanoparticles. Implanted ASC EVs or iEVs may attenuate inflammatory NFκB activity and inflammatory gene expression in injured tissue and promote tendon matrix regeneration in the early phase of tendon healing. Without being limited to a particular theory, the effects may be due to the ability of ASC EVs or iEVs in modulating tenocyte and macrophage activities. In some embodiments, ASC EVs or iEVs may reduce NFκB activity in injured tissue of the patient.

### EXAMPLES

### Example 1: Characterization of Mouse ASCs and ASC-Derived EVs

Mouse macrophages were derived from bone marrow of femurs and tibiae of adult NF-κB-GFP-luciferase (NGL) transgenic reporter mice for nuclear factor kappa-light-chain-enhancer of activated B cells (NF-κB) or wild type FVB/NJ (FVB) mice of both sexes and cultured in a macrophage culture medium containing 10% L929 cell conditioned medium (a source of macrophage colony stimulating factor), 100 unit/ml penicillin, 100 μg/ml streptomycin, and 10% fetal bovine serum in Minimum Essential Medium α. After 5 days, adherent cells were harvested and used for subsequent studies.

Mouse ASCs were isolated from the stromal vascular fraction of subcutaneous fat of adult ScxGFP or NGL mice of both sexes and expanded in ASC culture medium containing 10% FBS, 100 unit/ml penicillin, and 100 μg/ml streptomycin in α-MEM.

EVs were isolated from the conditioned medium of ASC culture. ASCs at passage 2-4 were primed with 100 ng/ml IFNγ overnight. The medium was subsequently removed. After three washes with sterile Dulbecco's phosphate buffered saline, the cells were further cultured in an EV collection medium (2% EV-free FBS in α-MEM) for 48 h. Conditioned medium from ASC culture (150 ml from approximately 2.5E+07 cells per isolation) with or without IFNγ pre-treatment was collected and centrifuged at 500 g for 10 min and 10,000 g for 30 min at 4° C. to remove large vesicles. After passing through a 0.22 μm filter, the medium was further centrifuged at 100,000 g for 90 min at 4° C. The resulting EV-free supernatant was collected as EV-free conditioned medium. The EV-containing precipitate was further washed and re-suspended in 70 μl DPBS. Some isolated ASC EVs were fluorescently labeled with PKH26. The EV-free FBS was prepared by ultracentrifugation of FBS at 100,000 g for 20 h to remove EVs in the FBS.

ASCs were assessed for their ability to form colony-forming units (CFUs), by their population doubling time, and by their surface marker expression with antibodies specific for MSC markers CD29, CD44, and CD90.

The size and concentration of isolated EVs were determined via either a Malvern Zen3600 Zetasizer or an Izon qNano Gold. EV protein concentrations were determined with a Thermo Scientific Micro BCA Protein Assay Kit. EV marker expression was determined by western blot with either rabbit anti-CD9 or rabbit anti-CD63 antibodies followed by HRP-conjugated goat-anti-rabbit secondary antibodies. Isolated ASC EVs were negatively stained with 1% aqueous uranyl acetate and viewed on a JEOL 1200EX transmission electron microscope.

Mouse ASCs exhibited a CFU frequency of approximately 1 in 300 and an average population doubling time of 1.68±0.74 days (N=5 isolations), similar to those reported in the literature. Over 90% of mouse ASCs expressed the MSC markers CD29, CD44 and CD90 (FIG. 2A). The ASCs with and without IFNγ priming yielded similar amounts of vesicles (1.61E+09±1.81E+09 and 1.67E+09±4.64E+08 particles/ml culture, respectively; N=3/group, p=0.995). The resulting iEVs and EVs were of similar size (mode diameter 108±2 nm and 113±3 nm) and were within the size range of exosomes. Transmission electron microscopy also showed that EVs and iEVs exhibited similar size and morphology (left and middle panels in FIG. 2B) and that the EV collection medium was free from EVs (right panel in FIG. 2B). Western blot further confirmed that ASC EVs expressed the exosome markers CD9 and CD63 (FIG. 2C), thus possessing the properties of exosome.

### Example 2: Determine the Role and Mechanisms of ASCs in Regulating Macrophage Polarization During Tendon Healing In Vitro and In Vivo

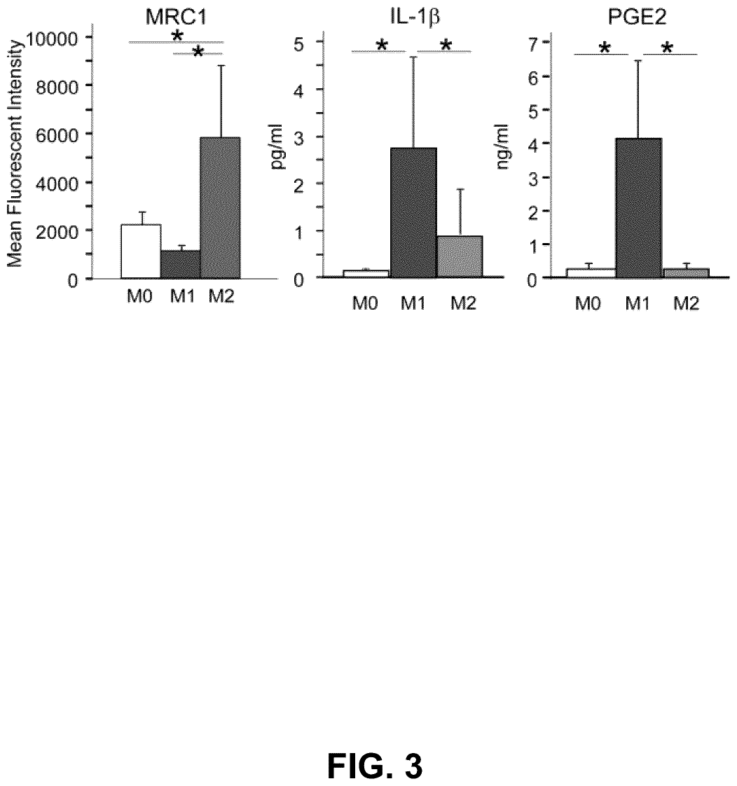

Macrophage polarization was induced and characterized. Mouse bone marrow-derived monocytes (M0) were induced into M1 and M2 macrophages by LPS+IFNγ and IL-4, respectively. As expected, among the three types of cells, the induced M2 macrophages expressed the highest level of a M2 marker MRC1, while the induced M1 macrophages produced the highest levels of IL1-β and PGE2 proteins (FIG. 3).

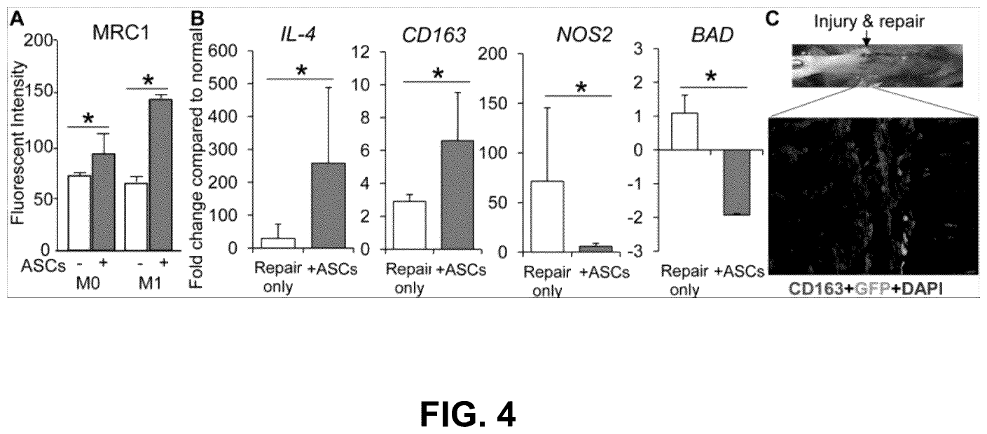

It was determined that ASCs facilitate M2 macrophages via a paracrine mechanism. In transwell culture, mouse ASCs induced the expression of the M2 marker MRC1 in macrophages in the absence (MO) or presence of M1 stimuli (M1; FIG. 4A). Consistently, in vivo study of a canine flexor tendon repair model revealed that ASCs, delivered in a form of thin sheet with collagen matrices, significantly increased the expression of a M2 stimulator gene IL-4 and a M2 marker gene CD163 in repaired tendons; meanwhile, the expression levels of a M1 marker gene NOS2 and an apoptotic gene BAD were reduced (FIG. 4B). Immunostaining further confirmed that ASCs (green in FIG. 4C) induced CD163+ cells (red in FIG. 4C) in their vicinity.

### Example 3: Determine the Role and Mechanisms of ASC EVs in Regulating Macrophage Inflammatory Response During Tendon Healing In Vitro

In vitro studies were conducted with EVs produced by mouse ASCs. The impact of ASC EVs on the macrophage inflammatory response was evaluated in EV-macrophage co-culture. Macrophages were stimulated with the pro-inflammatory cytokine interleukin-1β (IL-1β). IL-1β was chosen because it was the most significantly induced pro-inflammatory cytokine detected in the mouse Achilles tendon after injury and repair. To assess the EV-specific effect, EV collection medium (Control) and EV-free conditioned medium from ASC culture (Medium) were used as controls. The macrophage inflammatory responses were assessed via the NF-KB-luciferase reporter expressed by the NGL mice for the NF-KB-responsive luciferase activity.

NGL macrophages (30,000 cells/cm²) were pre-treated with either one of the following: Control, Medium, or EV for 24 h (N=3/condition). EVs were applied at a dose corresponding to an EV donor and recipient cell ratio of 20:1. The pre-treated cells were washed three times with DPBS. Macrophage NF-κB activity was subsequently determined in cell lysates 6 h after treatment with IL-1β (5 ng/ml) using a Dual-Luciferase Reporter Assay System.

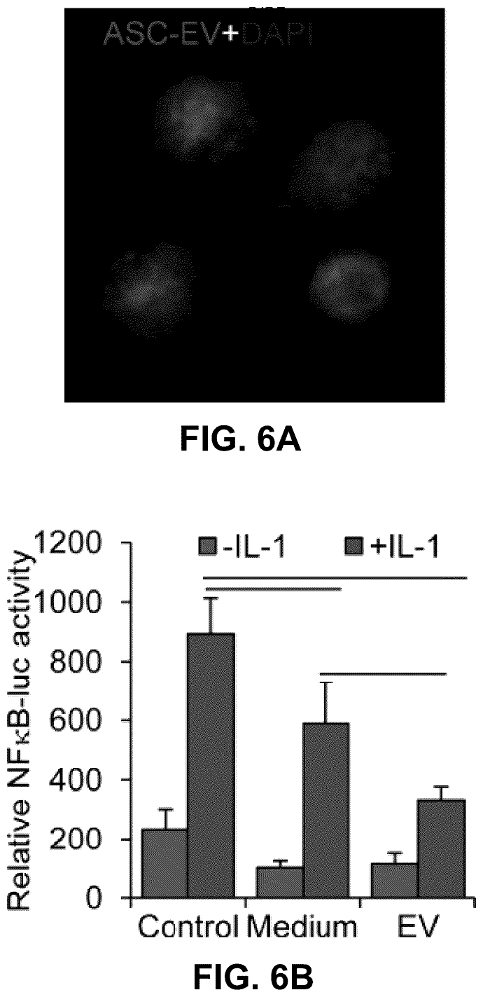

ASC EVs inhibit macrophage NFκB activity. As shown in FIG. 6A, mouse ASC EVs (in red) were picked up by macrophages (visualized via blue nuclear staining) and significantly inhibited the IL-1β-induced NFκB activity in macrophages (FIG. 6B. Control, Medium and EV, treated with EV-free medium, EV-free conditioned medium from ASCs and ASC EVs, respectively).

### Example 4: Determine the Clinically Relevant Efficacy of ASC EVs in Regulating Tendon Inflammation, NFκB Signaling in Particular, After Tendon Repair and Its Impact on Tendon Healing In Vivo

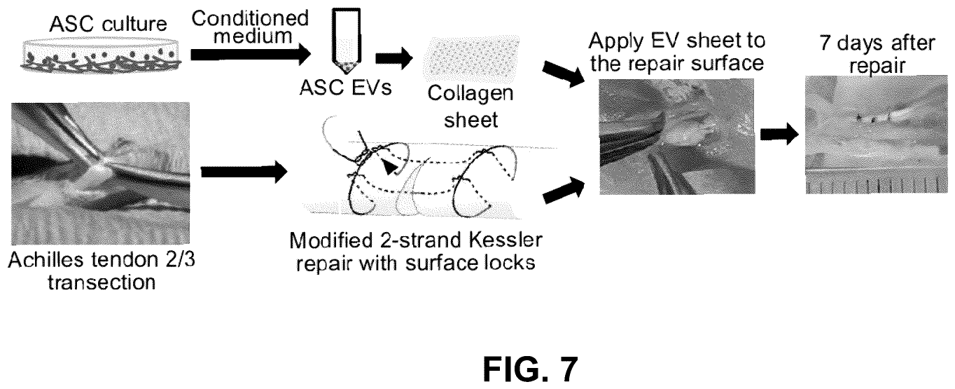

Achilles tendon 2/3 transection was conducted at the midpoint level between the calcaneal insertion and the musculotendinous junction of the right Achilles tendon. All transected tendons were repaired with a two-strand modified Kessler technique with surface locking followed by a simple peripheral suture (FIG. 7). Following repair, mice were allowed free movement after recovery from anesthesia.

A biocompatible thin collagen sheet was prepared and ASC EVs were loaded to the surface of the collagen sheet via their collagen binding properties. The EV-laden collagen sheet was cut into strips (2.5 mm×10 mm) that contained 5-6E+09 EVs from approximately one-half million ASCs and was applied around the repair site (FIG. 7). The EV dose was determined based on the ASC dose used previously. The distribution of PKH26-labeled ASC EVs in Achilles tendons was determined in 6-month old ScxGFP tendon reporter mice (N=2) 7 days after implantation by fluorescence microscopy. Vesicles were prelabeled with a red fluorescent dye PKH26.

To assess the impact of ASC EVs on the early tendon inflammatory response after injury, adult NGL mice of both sexes (3-4 months, weight 27±5 g) were used. After Achilles tendon partial transection and repair, the mice were randomly divided into three groups and received either of the following treatments: (i) collagen sheet only (Repair), (ii) collagen sheet loaded with EVs from naïve ASCs (+EV), and (iii) collagen sheet loaded with EVs from IFNγ-primed ASCs (+iEV). NF-κB activity at the repair site was determined in live mice via bioluminescence imaging at 1 day before (Pre) and 1, 3, and 7 days after (D1, D3, and D7) repair and treatment (N=4/group for Repair and +EV group, N=6 for +iEV group).

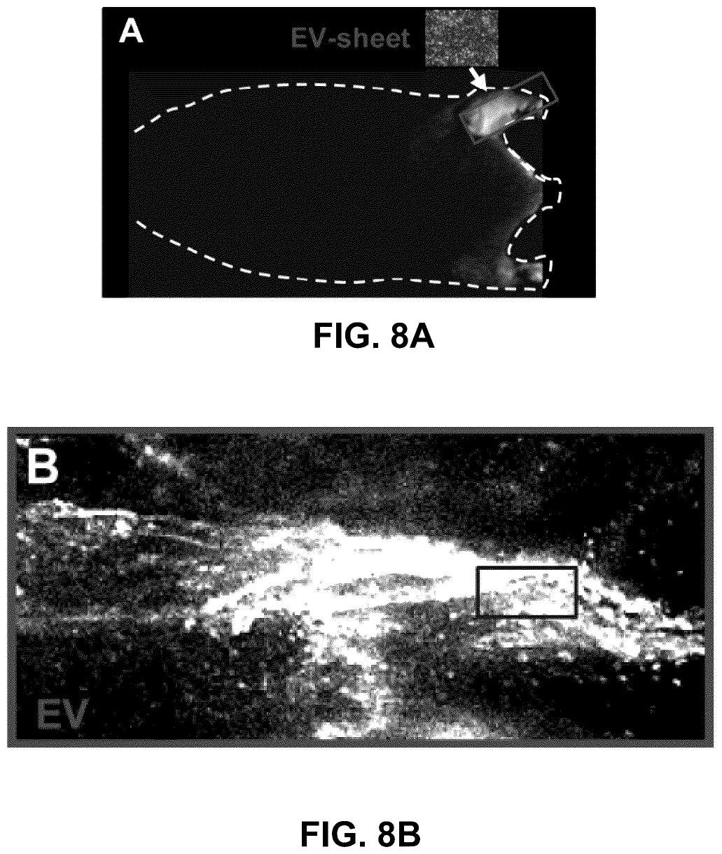

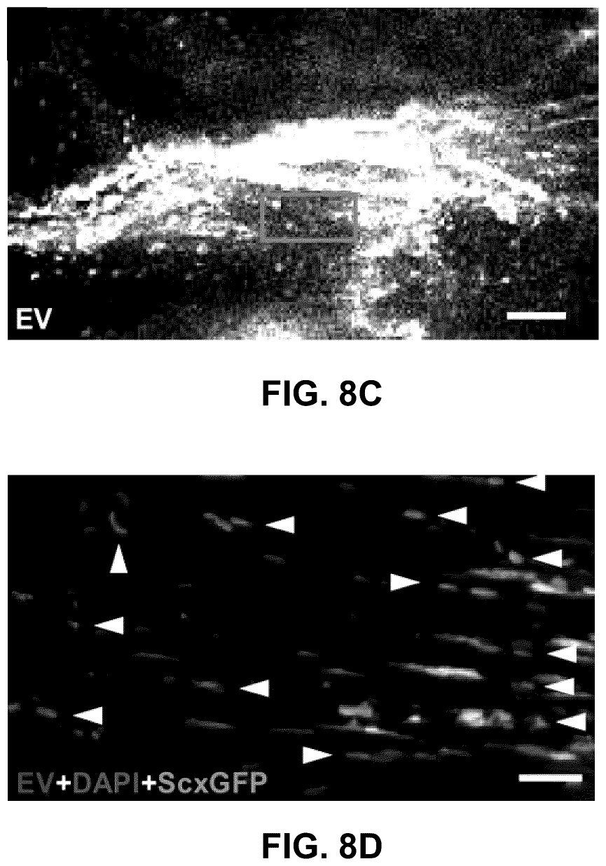

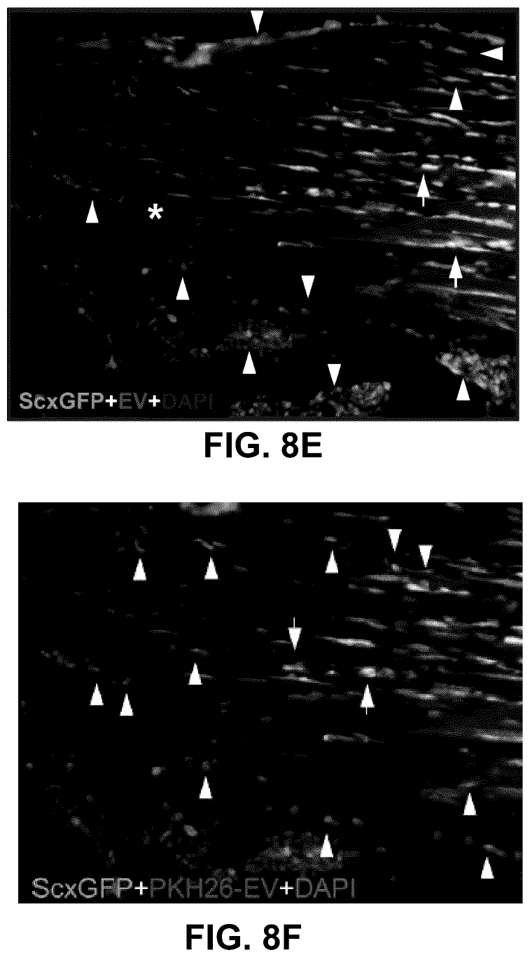

Implanted EVs were tracked via live fluorescence imaging (FIG. 8A). Local PKH26 signals were increased within the first a few days and remained stable at least until 7 days after repair. Whole mount fluorescence imaging confirmed intense fluorescent signals (in white color) from PKH 26-labeled EVs at the injury site along tendon fibers in ScxGFP tendon reporter mice 7 days after implantation (FIGS. 8B and 8C). Most of EV positive signals were co-localized with 4′,6-diamidino-2-phenylindole (DAPI) positive and ScxGFP negative cells (arrow heads in FIGS. 8D-8F), indicating ASC EVs primarily target infiltrating inflammatory cells at the injury center (asterisk). Some EVs were also found in fibroblast-like DAPI and ScxGFP double positive tenocytes near the injury center (arrows in FIGS. 8E and 8F).

Mice were injected intraperitoneally with D-luciferin (150 mg/kg in PBS) and imaged 10 min after injection under isoflurane anesthesia (2% vaporized in O₂) in an IVIS 50. Images were acquired with Living Image 4.3.1 software. Injury site total photon flux (photons/s) was measured from software-defined contour region of interest (ROI) that covers the injury site using Living Image 2.6 software. The result was normalized by the total photon flux of matching ROI of contralateral uninjured limb and expressed as a ratio of pre-injury level.

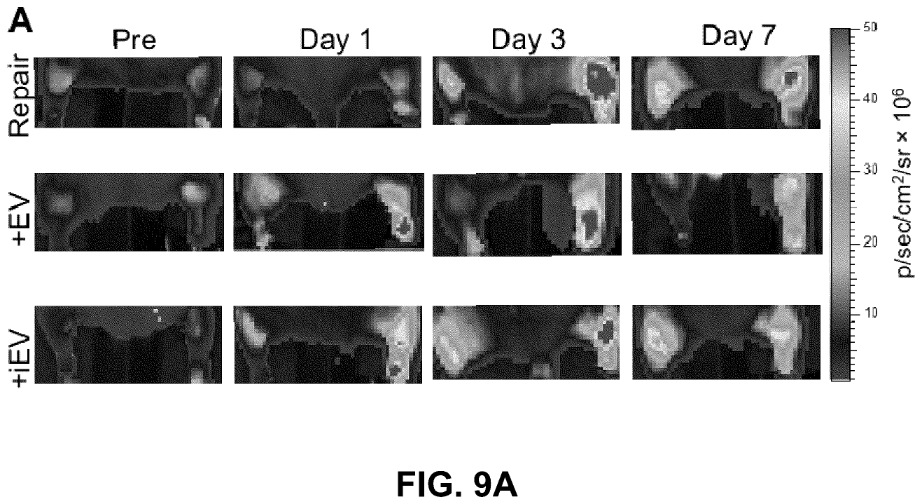

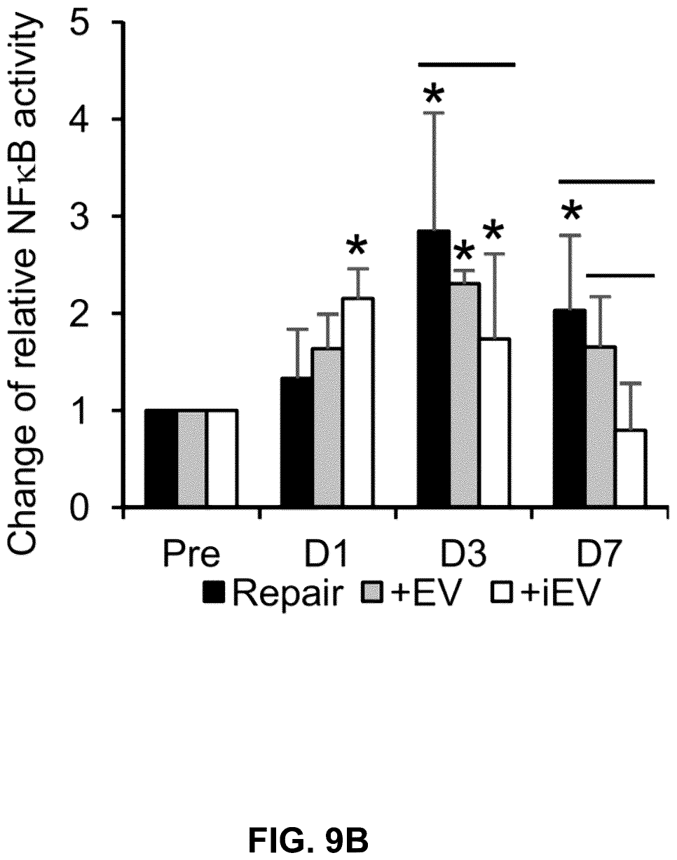

The effects of EVs and iEV on the repair site inflammatory response were assessed in the NGL NF-κB-luciferase reporter mice via live bioluminescence imaging (FIG. 9A). The live bioluminescence imaging revealed that Achilles tendon transection led to a substantial increase in NFκB activities at the injury site and application of EVs from primed but not naïve ASCs effectively attenuated the response (FIG. 9A). Specifically, NF-κB activity at the repair site of untreated tendons was dramatically increased at 3 days (FIG. 9B, p<0.001 vs. Pre) and 7 days (FIG. 9B, p=0.042 vs. Pre) following repair. By contrast, iEV-treated tendons showed significantly lower NF-κB activity compared to untreated tendons at 3 days (FIG. 9B, p=0.014) and 7 days (FIG. 9B, p=0.006) after repair. EV treatment did not alter the NF-κB activity in repaired tendons compared with the control repair (FIG. 9B; p=0.521, 0.193, and 0.364 vs. D1, D3, and D7, respectively). The NF-κB activity in EV-treated tendons was over twofold that of iEV-treated tendons at 7 days after repair (FIG. 9B, p=0.028 vs. +iEV).

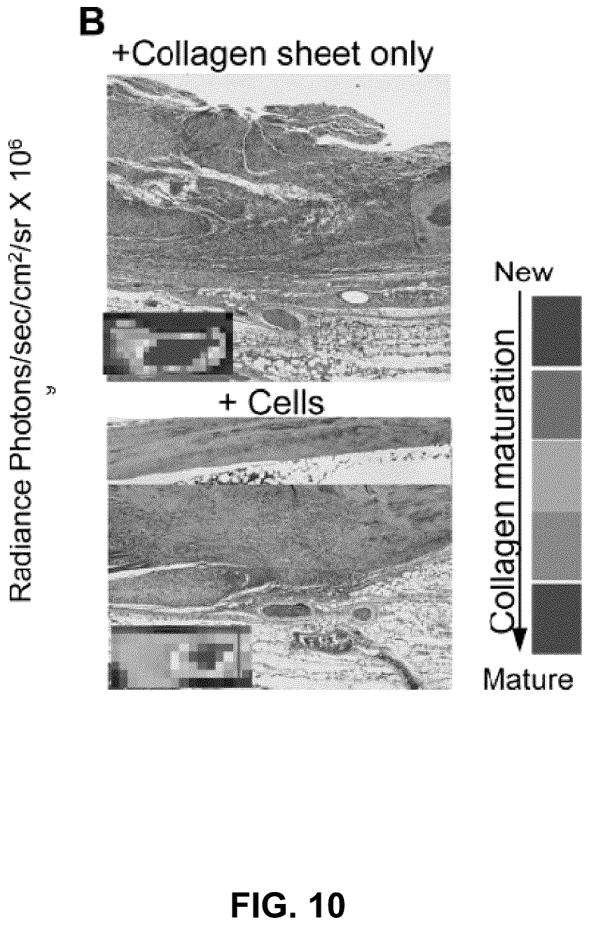

Tendon NFκB activity is inversely correlated with tendon healing response. Pentachrome staining allows for assessing collagen regeneration during tendon healing. As demonstrated in FIG. 10, attenuated NFκB-luc signals at the injury site, following a treatment with stem cells was accompanied with enhanced collagen synthesis/healing response.

### Example 5: Anti-Inflammatory and Pro-Regenerative Effects of Stem Cell-Derived Extracellular Vesicles in the Early Phase of Tendon Healing

After Achilles tendon partial transection and repair, the mice were randomly divided into three groups and treated with either of the followings: (i) collagen sheet only (Repair), (ii) collagen sheet loaded with EVs from naïve ASCs (+EV), and (iii) collagen sheet loaded with EVs from IFNγ-primed ASCs (+iEV). All mice were euthanized 7 days after repair. Repaired tendons were then dissected out for gene expression analysis (N=7/group).

Achilles tendons were pulverized with a Mikro-Dismembrator U and extracted in TRIzol Reagent. Total RNAs were isolated via phase separation using a Phase Lock Gel and purified with RNeasy MinElute Spin Columns. Five hundred nanograms of isolated total RNAs were reversely transcribed into cDNAs using a SuperScript IV VILO Master Mix. The relative abundances of genes of interest were determined by SYBR green real-time PCR using Qiagen or custom primers. Ipo8 was used as an endogenous reference gene. Changes in tendon gene expression were determined by the comparative Ct method and shown as fold changes relative to the expression levels in contralateral intact tendons. For genes that were near the detection limit in intact tendons, the results were reported as relative mRNA abundance (2−ΔCt).

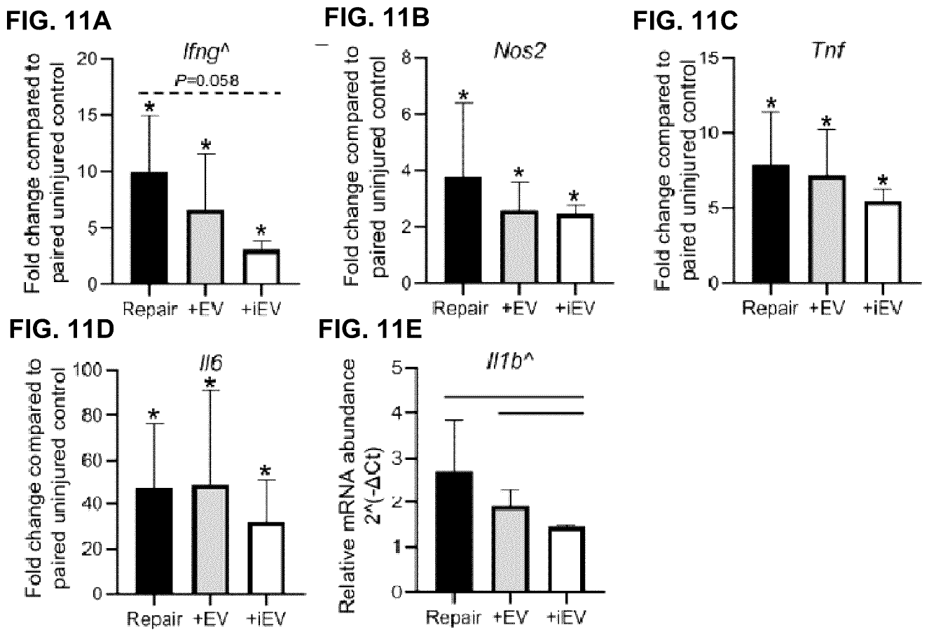

The effect of ASC EVs on the tendon inflammatory response was further assessed at the gene expression level in Achilles tendons 7 days after repair by RT-qPCR. In accordance with NF-κB activation, the expression levels of examined inflammatory genes Ifng, Nos2, Tnf, and II6 were all significantly increased after tendon injury and repair (FIGS. 11A-11D; p=0.000, 0.001, 0.000, and 0.005 vs. paired intact tendons, respectively). Moreover, II1b, which was barely detectable in intact tendons, became the most abundant inflammatory gene examined after tendon repair (FIG. 11E). Treatment with iEV but not EVs significantly reduced II1b expression in repaired ten-dons (FIG. 11E, p=0.007 vs. Repair and 0.011 vs. +EV). Similarly, significant differences in Ifng expression were detected among the three repair groups (FIG. 11A; \hat{ }p=0.045, one-way ANOVA) and iEVs but not EVs trended toward reducing Ifng expression after injury.

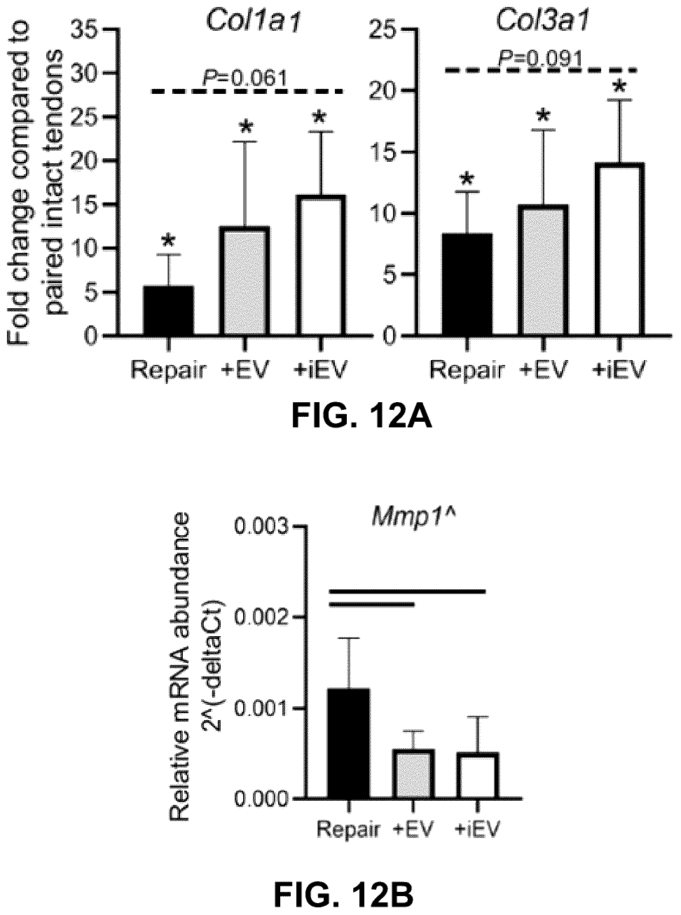

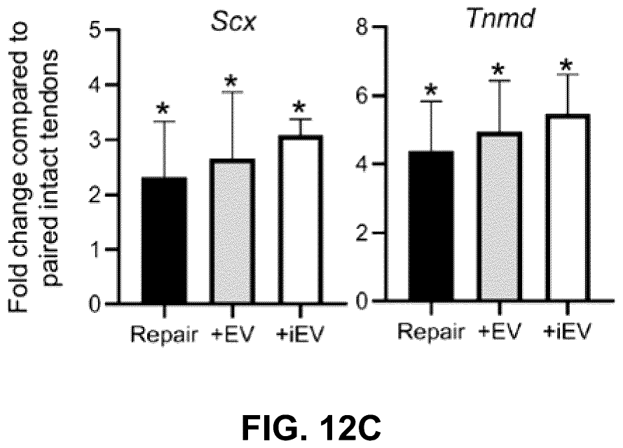

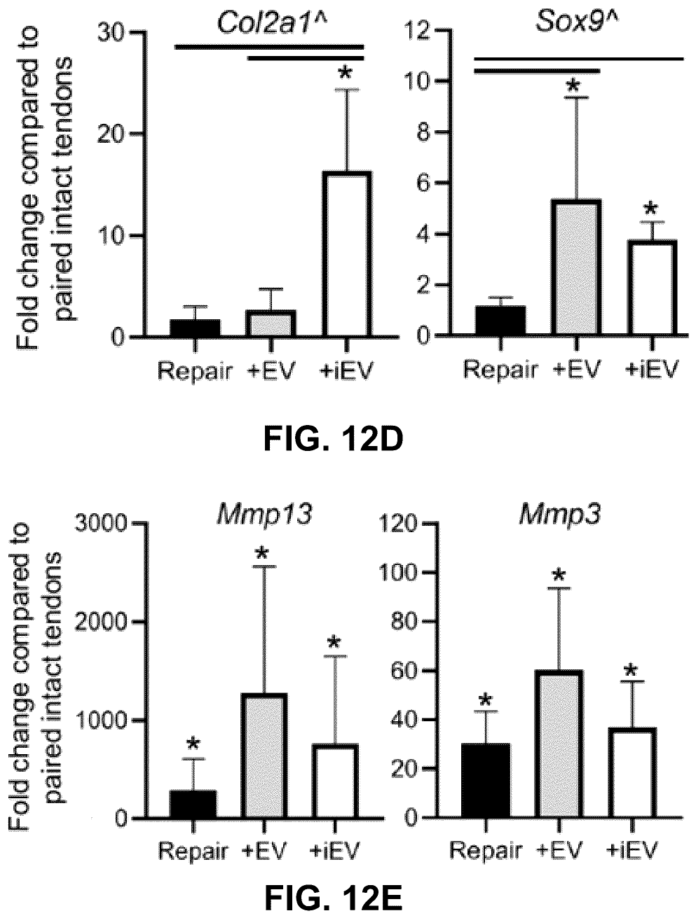

The expression levels of tendon matrix-related genes were also compared among three repair groups 7 days after injury. While Col1a1 and Col3a1 expression were increased in tendons from all three groups (FIG. 12A), Mmp1, a primary collagenase, which was undetectable in intact tendons, was induced after injury (FIG. 12B). Notably, treatment with both iEVs and EVs significantly attenuated the Mmp1 expression (FIG. 12A; \hat{ }p=0.007, one-way ANOVA; p=0.013 and 0.015, +EV and +iEV vs. Repair, respectively) and iEV but not EV treatment further trended toward increasing both Col1a1 and Col3a1 expression (FIG. 12A; p=0.057 and 0.092 for Col1a1 and Col3a1, respectively, by one-way ANOVA). The tenogenic genes Scx and Tnmd were also increased in tendons from all groups; whereas, no significant group differences were detected (FIG. 12C). In addition to Mmp1, tendon injury also triggered substantial increases in Mmp13 (764-fold) and Mmp3 (37-fold) expression in tendons from all three groups (FIG. 12E). Mmp13 and Mmp3 encode MMP13 and MMP3 proteins, respectively. Both MMP13 and MMP3 are collagenase that primarily breaks down type II collagen. While the expression level of Col2a1 that encodes the a chain of type II collagen was unchanged in untreated tendons but significantly increased in iEV-treated tendons after injury (FIG. 12D; p=0.002 and 0.013 vs. Repair and +EV, respectively); and iEVs and EVs also increased the expression of Sox9 that drives Col2a1 expression (FIG. 12D; p=0.005 and 0.009, +iEV and +EV vs. Repair, respectively). Therefore, the Sox9 and Col2a1 increases caused by iEV treatment that counteract the Mmp13 and Mmp3 increases after tendon injury may be also beneficial for tendon healing.

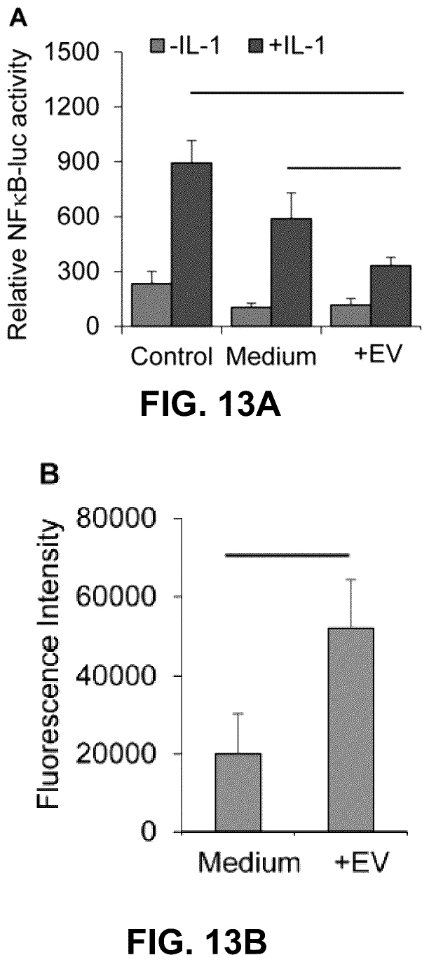

To explore the cellular basis of the observed EV effects, ASC EVs were co-cultured with macrophages and tenocytes, respectively, and evaluated for their effects on macrophage activity and tenocyte proliferation. Results revealed that ASC EVs were incorporated by both types of cells. A significant reduction in IL-1β-induced NFκB activity was noted in macrophages (FIG. 13A), in concert with an increase in tenocyte proliferation (FIG. 13B).

### Example 6: ASC iEVs Reduce Post-Operative Complications and Facilitate Anabolic Tissue Response After Tendon Injury

After Achilles tendon partial transection and repair, the mice were randomly divided into three groups and received either of the following treatments: (i) collagen sheet only (Repair, N=11), (ii) collagen sheet loaded with EVs from naïve ASCs (+EV, N=11), and (iii) collagen sheet loaded with EVs from IFNγ-primed ASCs (+iEV, N=10). All mice were euthanized 7 days after repair. Repaired tendons were surgically exposed as shown in FIG. 7. The integrity of repaired tendons was first assessed under a dissecting microscope. Post-operative gap formation and rupture were defined as partial and complete loss of the continuity of the repaired tendons, respectively. The assessed tendons were then dissected out for histological assessment (N=4, 3, and 3 for Repair, +EV, and +iEV group, respectively).

To assess tendon healing response histologically, Achilles tendons were fixed in 4% paraformaldehyde in PBS, embedded in paraffin, sectioned coronally at 5 um thickness, and stained with a pentachrome stain kit. Collagen in stained sections exhibits a bright red-orange color. The percentage of collagen-stained area in a 1.2 mm tendon fragment that covers the site of tendon injury was determined with the area analysis tool of Adobe Photoshop CC 2015.5.

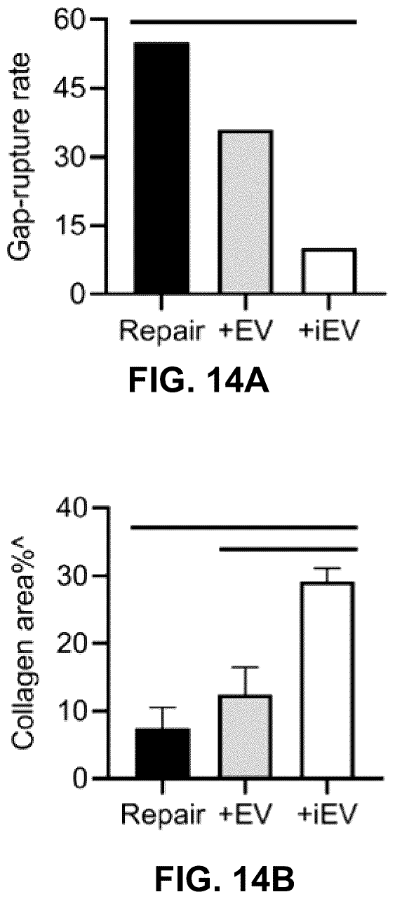

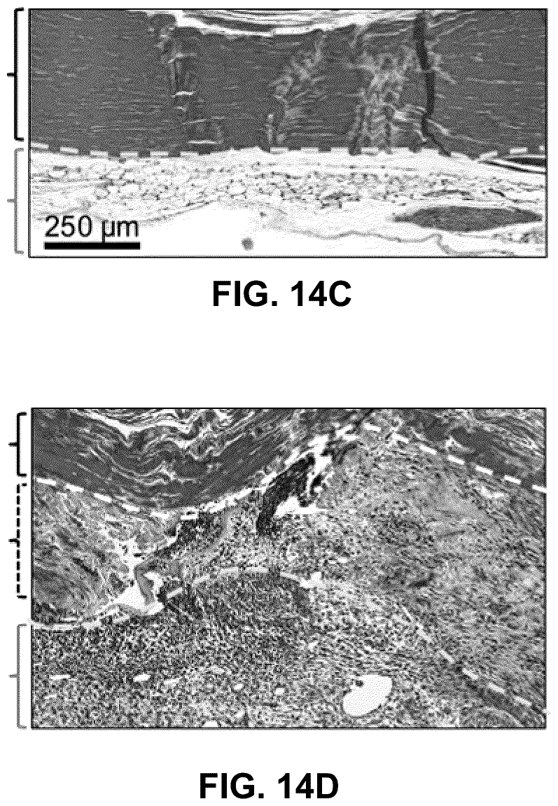

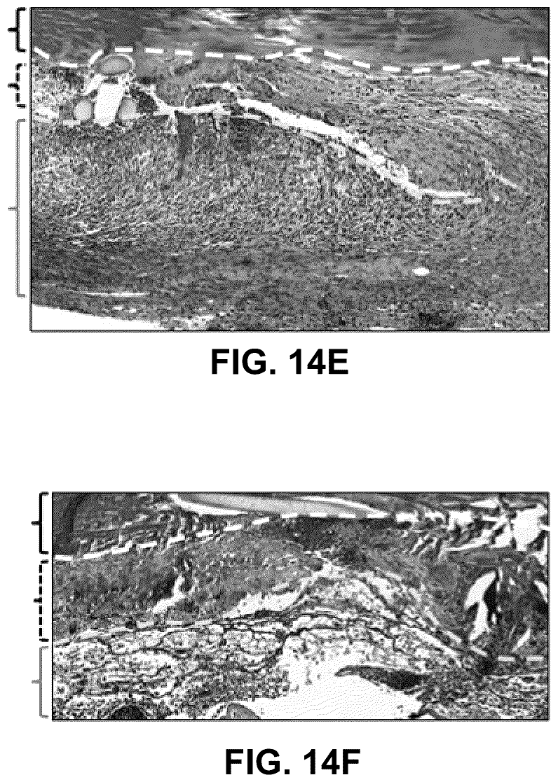

At the tissue level, iEV-treated tendons showed a much lower gap-rupture rate compared with untreated tendons (FIG. 14A; p=0.033 vs. Repair). Pentachrome staining on Achilles tendon sections revealed collagen in intact tendon in a bright red-orange color (FIG. 14C). The tendon is surrounded by a loose and fatty paratenon tissue (FIG. 14C, below a yellow dotted line). Tendon injury induced inflammatory cell infiltration and matrix deposition at the site of tendon injury (between the two dotted lines in FIGS. 14D-14F) and within the adjacent paratenon (FIGS. 14D-14F, below the yellow dotted lines). While we did not detect consistent histological differences in the cellularity and vascularity among tendons from the different repair groups, the iEV-treated tendons exhibited more collagen staining at the site of tendon injury than did untreated and EV-treated tendons (FIG. 14B, p<0.001 and p=0.004 vs. Repair and +EV, respectively; FIGS. 14D-14F).

### Example 7: ASC iEVs are More Effective Than EVs in Suppressing NF-γB Activation in Macrophages

In vitro studies were conducted with EVs produced by IFNγ-primed and naïve mouse ASCs (iEVs and EVs). The impact of EVs and iEVs on the macrophage inflammatory response was compared in EV-macrophage co-culture. Macrophages were stimulated with the pro-inflammatory cytokine interleukin-1β (IL-1β). IL-1β was chosen because it was the most significantly induced pro-inflammatory cytokine detected in the mouse Achilles tendon after injury and repair. To assess the EV-specific effect, EV collection medium (medium) and EV-free conditioned medium from naïve and primed ASC culture (CM and iCM) were used as controls. The macrophage inflammatory responses were assessed via the NF-κB-luciferase reporter expressed by the NGL mice for the NF-κB-responsive luciferase activity and luciferase mRNA expression. All results were obtained from at least three independent experiments.

NGL macrophages (30,000 cells/cm²) were pre-treated with either one of the following: medium, CM, iCM, EV, or iEV for 24 h (N=3-4/condition in duplicate). EVs and iEVs were applied at a dose corresponding to an EV donor and recipient cell ratio of 20:1. The dose was determined based on a previously published study. The pre-treated cells were washed three times with DPBS. Macrophage NF-κB activity was subsequently determined in cell lysates 6 h after treatment with IL-162 (5 ng/ml) using a Dual-Luciferase Reporter Assay System. The results were normalized by the protein concentrations of respective samples. Macrophage gene expression was assessed at 24 h after IL-1β (10 ng/ml) treatment in cell lysates by a SYBR green-based quantitative real-time polymerase chain reaction (qPCR).

To determine if the observed in vivo effects of ASC EVs and iEVs resulted from their differential ability to modulate the macrophage inflammatory response. NGL macro-phages were co-cultured with either EVs or iEVs prelabeled with PKH26 for 24 h. Live fluorescence imaging detected PKH26 signals in nearly all cells without apparent differences between EVs (FIG. 15A) and iEVs (FIG. 15B). Moreover, a comparison between NGL (FIGS. 15A and 15B) and wild type FVB macrophages (FIG. 15C) showed that macrophages with and without the NGL transgene took EVs with similar efficacy and that the EVs taken by either NGL or FVB macrophages were commonly localized to the cell bodies and not the protrusions. FIGS. 15A-15C are representative superimposed fluorescence and bright field images of isolated NF-κB-GFP-luciferase (NGL) (FIGS. 15A-15B) and FVB (FIG. 15C) macrophages co-cultured with fluorescently labeled EVs (shown in white in FIGS. 15A and 15C) or iEVs (white in FIG. 15B).

The effects of EVs and iEVs on IL-1β-induced NF-κB activity in NGL macrophage were determined via a luciferin/luciferase-based assay. As shown in FIG. 15D both EVs and iEVs but not CM or iCM blocked the IL-1β-induced NF-κB activity in macrophages (FIG. 15D; p=0.114, 0.276, 0.006, and 0.005, +CM, +iCM, +EV, and +iEV vs. Medium, respectively). Unexpectedly, while EVs were less effective than iEVs in vivo, the two treatments were equally effective in vitro. This difference may be due to a more extreme inflammatory environment present in vivo, which requires a more potent anti-inflammatory agent. To test this concept, the concentration of IL-1β was increased from 5 to 10 ng/ml and the effects of EVs and iEVs were further compared for the NF-κB-responsive luciferase transgene expression by the more sensitive RT-qPCR. The result confirmed that, while both EV and iEV were effective in reducing IL-1β-induced NF-κB activity in macrophages (FIG. 15E; p=0.008 and p<0.0001, +EV and +iEV vs. Medium, respectively), iEVs were nearly three times more effective than EVs (p=0.004, +iEV vs. +EV).

The particular variations disclosed above are illustrative only, as the variations may be modified and practiced in different but equivalent manners apparent to those skilled in the art having the benefit of the teachings herein. It is therefore evident that the particular variations disclosed above may be altered or modified, and all such variations are considered within the scope and spirit of the application. Accordingly, the protection sought herein is as set forth in the description. Although the present variations are shown above, they are not limited to just these variations, but are amenable to various changes and modifications without departing from the spirit thereof. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed variations teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1.-8. (canceled)

9. A method of preparing a tissue repair matrix comprising:
harvesting a plurality of ASCs;
culturing and inducing the harvested ASCs;
isolating a plurality of EVs from the ASCs; and
loading a collagen sheet with the plurality of EVs.

10. The method of claim 9, further comprising priming the ASCs with inflammatory cytokines.

11. A method for treating an injured tissue comprising applying a plurality of bionanoparticles to the injured tissue wherein the plurality of bionanoparticles comprise at least one isolated extracellular vesicle (iEV) from IFN-γ primed adipose-derived mesenchymal stromal cells (ASCs); microRNA and/or mRNA within the iEV, and wherein the microRNA and/or mRNA inhibits the macrophage inflammatory response and/or modulates tendon cell proliferation and collagen production.

12. (canceled)

13. The method of claim 11, wherein the bionanoparticles are applied directly to the injured tissue.

14. The method of claim 11, wherein the bionanoparticles are injected peritendinously, subcutaneously, or intra-articularly near the injured tissue.

15. The method of claim 11, wherein the injured tissue is musculoskeletal tissue or soft tissue.

16. The method of claim 15, wherein musculoskeletal tissue is a tendon or ligament selected from an Achilles tendon, patellar tendon, rotator cuff tendon, or flexor tendon.

17. The method of claim 11, wherein the tissue repair matrix is applied during operative repairs.

18. The method of claim 11, wherein the tissue repair matrix is placed on top the injured tissue, surrounds the injured tissue, and/or is attached to the injured tissue with or without suturing.

19. The method of claim 11, wherein the ASC EVs attenuate inflammatory NF-KB activity in the injured tissue.

20. The method of claim 11, wherein the ASC EVs promote tendon matrix regeneration during tendon healing.
